(19) United States
(12) Patent Application Publication
(10) Pub. No.: US 2026/0148818 A1
(43) Pub. Date: May 28, 2026

(54) X-RAY CT APPARATUS, MEDICAL INFORMATION PROCESSING SYSTEM, AND MEDICAL INFORMATION PROCESSING METHOD

(71) Applicant: Canon Medical Systems Corporation, Otawara-shi (JP)

(72) Inventors: Hideyuki SUZUKI, Nasushiobara (JP); Junichi TAKAZAWA, Nasushiobara (JP); Hiroki SAITO, Oyama (JP); Nobuki MOROOKA, Nasushiobara (JP)

(73) Assignee: Canon Medical Systems Corporation, Otawara-shi (JP)

(21) Appl. No.: 19/396,551

(22) Filed: Nov. 21, 2025

(30) Foreign Application Priority Data
Nov. 22, 2024 (JP) 2024-204146

(51) Int. Cl. G16H 15/00; A61B 6/00; A61B 6/03; G16H 10/60

(52) U.S. Cl. CPC G16H 15/00; A61B 6/032; A61B 6/563; G16H 10/60

(57) ABSTRACT

According to one embodiment, an X-ray CT apparatus includes a processing circuit. The processing circuit analyzes history information concerning a radiogram interpretation report. The processing circuit updates rule information for outputting warning information concerning correction of the radiogram interpretation report based on an analysis result of the history information.

### CROSS-REFERENCE TO RELATED APPLICATIONS

This application is based upon and claims the benefit of priority from prior Japanese Patent Application No. 2024-204146, filed Nov. 22, 2024, the entire contents of which are incorporated herein by reference.

### FIELD

Embodiments described herein relate generally to an X-ray CT apparatus, a medical information processing system, and a medical information processing method.

### BACKGROUND

Conventionally, there has been known a technique of detecting an inappropriate description by analyzing a radiogram interpretation report and analyzing the descriptions of the radiogram interpretation report using rule information set by an operator or the like and notifying the radiogram interpretation doctor of the inappropriate description.

### BRIEF DESCRIPTION OF THE DRAWINGS

FIG. 1 is a block diagram showing an arrangement example of a medical information processing system according to a first embodiment.

FIG. 2 is a block diagram showing an arrangement example of a radiogram interpretation report preparation apparatus according to the first embodiment.

FIG. 3 is a block diagram showing an arrangement example of a radiogram interpretation report preparation support apparatus according to the first embodiment.

FIG. 4 is a block diagram showing an arrangement example of a feedback apparatus according to the first embodiment.

FIG. 5 is a block diagram showing an arrangement example of a linkage with a medical treatment system according to the first embodiment.

FIG. 6 is a view showing an example of screen display of a radiogram interpretation apparatus according to the first embodiment.

FIG. 7 is a view showing an example of a warning rule database of rule information according to the first embodiment.

FIG. 8 is a view showing an example of rule sections according to the first embodiment.

FIG. 9 is a view showing an example of display history information according to the first embodiment.

FIG. 10 is a view showing an example of the correction history information of a radiogram interpretation report according to the first embodiment.

FIG. 11 is a view showing an example of operation history information according to the first embodiment.

FIG. 12 is a view showing an example of the database of rule information concerning left-right mix-ups according to the first embodiment.

FIG. 13 is a view showing an example of the database of rule information concerning sex according to the first embodiment.

FIG. 14 is a view showing an example of the database of rule information concerning the notification timings at which warning information is output according to the first embodiment.

FIG. 15 is a view showing an example of the database of rule information concerning a linkage with a medical treatment system according to a second embodiment.

FIG. 16 is a flowchart for the addition of rule information according to the first embodiment.

FIG. 17 is a flowchart for the deletion of rule information with the display of warning information according to the first embodiment.

FIG. 18 is a flowchart for the deletion of rule information without the display of warning information according to the first embodiment.

FIG. 19 is a block diagram showing an arrangement example of an X-ray CT apparatus according to a third embodiment.

### DETAILED DESCRIPTION

In general, according to one embodiment, an X-ray CT apparatus includes a processing circuit. The processing circuit analyzes history information concerning a radiogram interpretation report. The processing circuit updates rule information for outputting warning information concerning correction of the radiogram interpretation report based on an analysis result of the history information.

A medical information processing apparatus, a radiogram interpretation report preparation apparatus, a medical information processing system, a medical information processing program, and a medical information processing method will be described in detail below with reference to the accompanying drawings. The same reference numerals denote components that perform similar operations, and redundant descriptions will be omitted as appropriate. The medical information processing apparatus, the radiogram interpretation report preparation apparatus, the medical information processing system, the medical information processing program, and the medical information processing method are not limited to the embodiments described below.

### First Embodiment

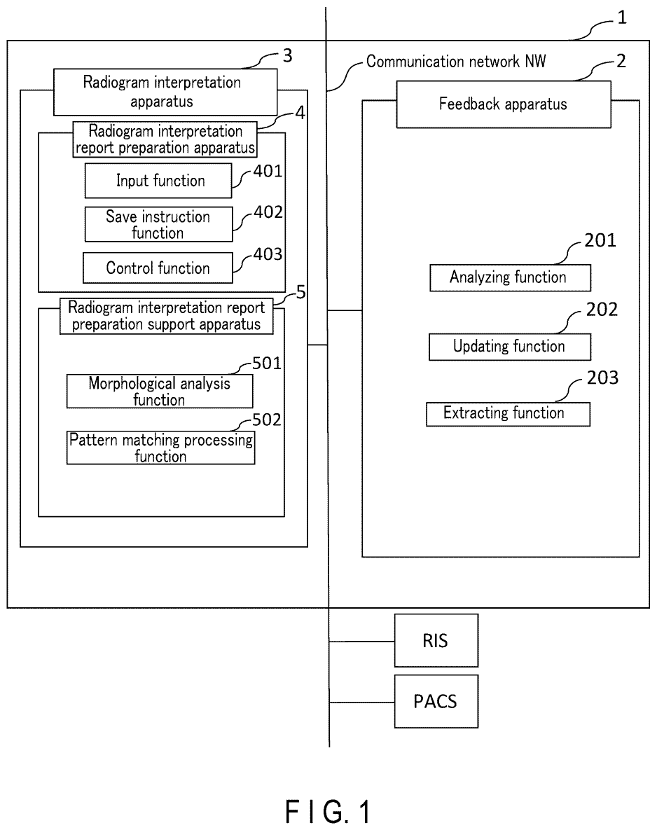

The overall arrangement of a medical information processing system 1 according to an embodiment will be described. FIG. 1 shows an arrangement example of the medical information processing system 1 according to the present embodiment. For example, as shown in FIG. 1, a feedback apparatus 2 according to the present embodiment is connected to a radiogram interpretation report preparation apparatus 4 and a radiogram interpretation report preparation support apparatus 5 via a communication network NW. A radiogram interpretation apparatus 3 is implemented by combining the radiogram interpretation report preparation apparatus 4 and the radiogram interpretation report preparation support apparatus 5.

The radiogram interpretation report preparation apparatus 4 is an apparatus for allowing a radiogram interpretation doctor to prepare a radiogram interpretation report. The radiogram interpretation report preparation apparatus 4 receives examination order information from a RIS (Radiology Information System) and updates examination information 406. The radiogram interpretation report preparation apparatus 4 receives an examination image from a PACS (Picture Archiving and Communication System) and updates the examination information 406. The radiogram interpretation doctor prepares a radiogram interpretation report by using the examination order information and the examination image held by the examination information 406. The radiogram interpretation report preparation apparatus 4 transmits the prepared radiogram interpretation report to a report DB 404.

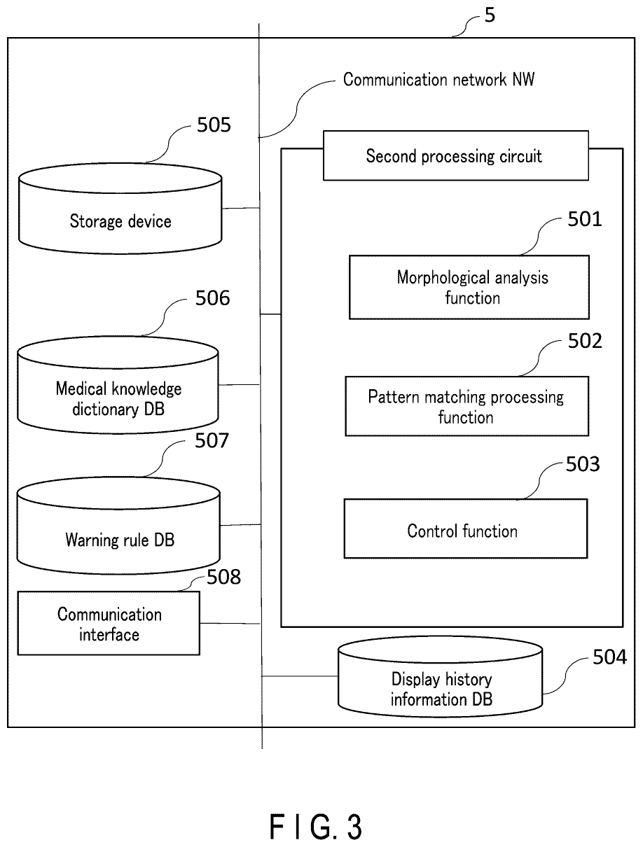

The radiogram interpretation report preparation support apparatus 5 is implemented by a combination of element technologies including morphological analysis, a medical knowledge dictionary, and pattern matching processing. The radiogram interpretation report preparation support apparatus 5 is an apparatus that analyzes the descriptions of a radiogram interpretation report based on a warning rule DB 507 (FIG. 3) and displays a warning message on a screen of the radiogram interpretation report preparation apparatus upon detecting an inappropriate description to prompt the radiogram interpretation doctor to correct the inappropriate description. Inappropriate descriptions include an ambiguous expression, an expression that tends to lead to a misunderstanding, a contradictory expression, and a mistake. Morphological analysis is performed to decompose a document in a finding column of a radiogram interpretation report into meaningful units and specify a part of speech for each decomposed unit. Pattern matching processing is performed to perform collation with the medical knowledge dictionary for each part of speech specified by morphological analysis.

The radiogram interpretation report preparation support apparatus 5 performs, for example, notational analysis on Japanese, analysis on radiogram interpretation terms, presence analysis on descriptions about an examination purpose, previous report reference analysis, and analysis on the order of finding descriptions. Notational analysis on Japanese is performed to analyze, for example, typing mistakes and term differences. Notational analysis is not limited to Japanese and may be performed on other languages such as English and Chinese. Analysis on radiogram interpretation terms is performed to analyze, for example, differences in disease name, examination technique name, and symptom term. Presence analysis on descriptions about an examination purpose is performed to analyze, for example, whether a description about an examination purpose region or symptom is insufficient. Examination order information held in the examination information 406 in the radiogram interpretation report preparation apparatus 4 includes an examination purpose. A control function 503 of the radiogram interpretation report preparation support apparatus 5 acquires an examination purpose from the server of the radiogram interpretation report preparation apparatus 4 and stores the examination purpose in a storage device 505 of the radiogram interpretation report preparation support apparatus 5. A pattern matching processing function 502 of the radiogram interpretation report preparation support apparatus 5 refers to the examination purpose region and symptom stored in the storage device 505, expressions written in a radiogram interpretation report by a morphological analysis function 501, and a medical knowledge dictionary DB 506 to analyze whether the radiogram interpretation report is insufficient. Previous report reference analysis is performed to analyze, for example, whether there is any description about comparison with a previous radiogram interpretation result at the time of follow-up. Analysis on a finding description order is performed to analyze, for example, whether an important finding is written first. In general, a radiogram interpretation doctor performs radiogram interpretation from an upper part to a lower part of the body when setting the body trunk as a radiogram interpretation target and inputs a finding to a radiogram interpretation report. Meanwhile, the radiogram interpretation doctor describes an answer to the examination purpose in an upper portion of the radiogram interpretation report. That is, an important finding is, for example, a finding concerning an answer corresponding to an examination purpose. The severities of diseases are managed in the medical knowledge dictionary DB 506. The pattern matching processing function 502 of the radiogram interpretation report preparation support apparatus 5 refers to severities. If the severity of a given disease is high, the radiogram interpretation report preparation support apparatus 5 analyzes whether a finding concerning the disease with high severity is written in an upper portion of the radiogram interpretation report.

When a save instruction function 402 of the radiogram interpretation report preparation apparatus 4 saves the radiogram interpretation report prepared by the radiogram interpretation doctor, the control function 503 of the radiogram interpretation report preparation support apparatus 5 stores the radiogram interpretation report saved in the radiogram interpretation report preparation apparatus 4 in the storage device 505 of the radiogram interpretation report preparation support apparatus 5 via a communication interface 508. A control function 403 acquires information concerning the display portion of an item as a warning target in the radiogram interpretation report and a warning message as a result of analysis performed by the second processing circuit of the radiogram interpretation report preparation support apparatus 5 and transmits the information concerning the display portion of the item as the warning target in the radiogram interpretation report and the warning message to a storage device 405 of the radiogram interpretation report preparation apparatus 4 via the communication interface 508. The control function 403 of the radiogram interpretation report preparation apparatus 4 controls a display 410 to display the display portion of the item as the warning target in the radiogram interpretation report and the warning message stored in the storage device 405 and the warning message on the display screen on which the radiogram interpretation report is displayed. Note that this processing is not limited to the timing at which a radiogram interpretation report is saved. For example, the processing may be performed when the radiogram interpretation report preparation apparatus 4 is activated, when a key image is pasted on a radiogram interpretation report, when the result of examination on a patient is imported, when a speech input is confirmed with respect to a finding of a radiogram interpretation report, or at arbitrary date and time set by an operator. Alternatively, the control function 403 of the radiogram interpretation report preparation apparatus 4 may notify the radiogram interpretation doctor by speech of the display portion of the item, stored in the storage device 405, as the warning target of the radiogram interpretation report and the warning message. In addition, the display portion of the item as the warning target may be part of the finding or part of information in the item. To output warning information concerning the correction of a radiogram interpretation report is to, for example, display the warning message or notify it by speech.

The feedback apparatus 2 updates the warning rule DB 507 of the radiogram interpretation report preparation support apparatus 5. Updating is, for example, adding, deleting, or changing. The feedback apparatus 2 analyzes the history information of each apparatus, for example, the warning history information displayed on the radiogram interpretation report preparation apparatus 4 and adds, deletes, or changes rule information in the warning rule DB 507.

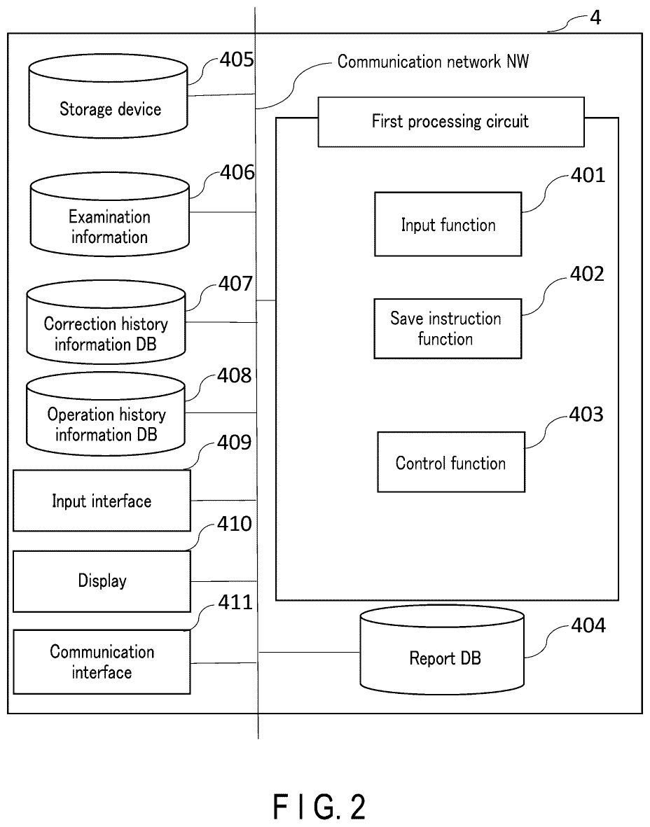

FIG. 2 is a block diagram showing an arrangement example of the radiogram interpretation report preparation apparatus 4 according to the present embodiment. As shown in FIG. 2, the radiogram interpretation report preparation apparatus 4 includes the first processing circuit, the storage device 405, the examination information 406, a correction history information DB 407, an operation history information DB 408, the report DB 404, an input interface 409, the display 410, and a communication interface 411.

The first processing circuit includes a CPU (Central Processing Unit) and a processor such as a GPU (Graphics Processing Unit). To prepare a radiogram interpretation report, the first processing circuit implements an input function 401, the save instruction function 402, the control function 403, and the like by causing the processor to activate the radiogram interpretation report preparation program installed in the storage device 405 or the like. Note that the functions 401 to 403 each need not always be implemented by a single processing circuit. Each processing circuit may be formed by combining a plurality of independent processors, and each processor may implement each of the functions 401 to 403 by executing a program. Alternatively, the input function 401, the save instruction function 402, and the control function 403 each may be a program as a module constituting a radiogram interpretation preparation program or may be an individual program. These programs are stored in the storage device 405.

The storage device 405 is a storage device such as a ROM (Read Only Memory), RAM (Random Access Memory), HDD (Hard Disk Drive), SSD (Solid State Drive), or integrated circuit storage device that stores various types of information. Other than the above storage device, the storage device 405 may be a driver that reads and writes various types of information between a portable storage medium such as a CD (Compact Disc), DVD (Digital Versatile Disc), or flash memory and a semiconductor storage device. Alternatively, the storage device 405 may be placed in another computer connected to the radiogram interpretation report preparation apparatus 4 via the communication network NW. A storage device 204 (to be described later) and the storage device 505 each have the same arrangement.

An examination list is a list that updates information indicating whether the preparation of a radiogram interpretation report about examination order information has been executed. The radiogram interpretation doctor selects a radiogram interpretation target from the radiogram interpretation list and prepares a radiogram interpretation report. The examination information 406 includes examination order information and an examination list.

The input interface 409 receives various types of input operations from the operator, converts the received input operations into electrical signals, and outputs the electrical signals to the first processing circuit. More specifically, the input interface 409 is connected to input devices such as a mouse, a keyboard, a trackball, switches, buttons, a joystick, a touch pad, and the touch panel display 410. The input interface 409 outputs an electrical signal corresponding to an input operation on the input device to the first processing circuit. An input device connected to the input interface 409 may be an input device provided for another computer connected via the communication network NW.

The display 410 displays various types of information in accordance with the control function 403 of the first processing circuit. For example, the display 410 displays a radiogram interpretation report preparation screen and an extracted item display window. As the display 410, for example, it is possible to use a liquid crystal display (an LCD: Liquid Crystal Display), a CRT (Cathode Ray Tube) display, an organic EL display (OELD: Organic Electro Luminescence Display), a plasma display, or another arbitrary display as appropriate. In addition, a projector may be provided in place of or in addition to the display 410.

The communication interface 411 is an interface for data communication of various types of medical information with another computer of a HIS (Hospital Information System), a RIS (Radiology Information System), a PACS, or a medical image diagnosis apparatus. For example, the communication interface 411 receives medical images from the medical image diagnosis apparatus and the PACS.

The correction history information DB 407 is a database that stores history information having undergone correction such as writing error correction, finding addition, finding deletion, or key image re-pasting by the radiogram interpretation doctor upon checking warning information. The correction history information DB 407 may be a log that records and accumulates events in chronological order.

The operation history information DB 408 is a database storing history information indicating operations until the display of warning information on a display screen of the radiogram interpretation report preparation apparatus 4, such as activating the viewer 7, pasting a key image, and receiving an examination result. The operation history information DB 408 is not limited to a database and may be a log that records and accumulates events in chronological order.

The report DB 404 is a database that saves the radiogram interpretation report prepared by the radiogram interpretation doctor.

FIG. 3 is a block diagram showing an arrangement example of the radiogram interpretation report preparation support apparatus 5 according to the present embodiment. As shown in FIG. 3, the radiogram interpretation report preparation support apparatus 5 includes the second processing circuit, the storage device 505, the medical knowledge dictionary DB 506, the warning rule DB 507, a display history information DB 504, and the communication interface 508.

The second processing circuit includes a CPU and a processor such as a GPU. In order to analyze an inappropriate description in a prepared radiogram interpretation report, the second processing circuit causes the processor to activate the radiogram interpretation report preparation support program installed in the storage device 505 or the like to implement the morphological analysis function 501, the pattern matching processing function 502, the control function 503, and the like. Note that the functions 501 to 503 each need not always be implemented by a single processing circuit. Each processing circuit may be formed by combining a plurality of independent processors, and each processor may implement each of the functions 501 to 503 by executing a program. Alternatively, the morphological analysis function 501, the pattern matching processing function 502, and the control function 503 each may be a program as a module constituting the radiogram interpretation preparation program or may be an individual program. These programs are stored in the storage device 505.

The medical knowledge dictionary DB 506 is a collection of medical term knowledge and conceptual relationship knowledge. For example, the medical knowledge dictionary DB 506 is configured by expressions such as formal expressions of writing errors and abbreviations, wrong medical terms, comparative information with respect to previous radiogram interpretation results, and information corresponding to inappropriate description orders.

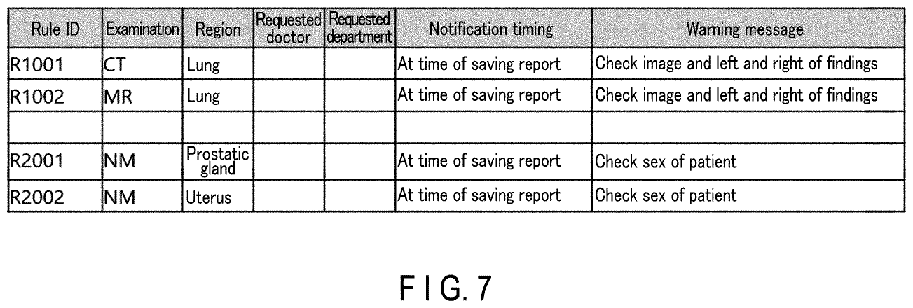

The warning rule DB 507 is a relational database by configured by a table like that shown in FIG. 7. The configuration of the database is not limited to a relational database. For example, the configuration may be a hierarchical database or network-type database. Each rule information corresponds to a medical knowledge dictionary. The morphological analysis function 501 decomposes a document into minimum meaningful units and specifies a part of speech for each decomposed unit. The pattern matching processing function 502 performs collation with the medical knowledge dictionary DB 506 for each part of speech decomposed and specified by the morphological analysis function 501.

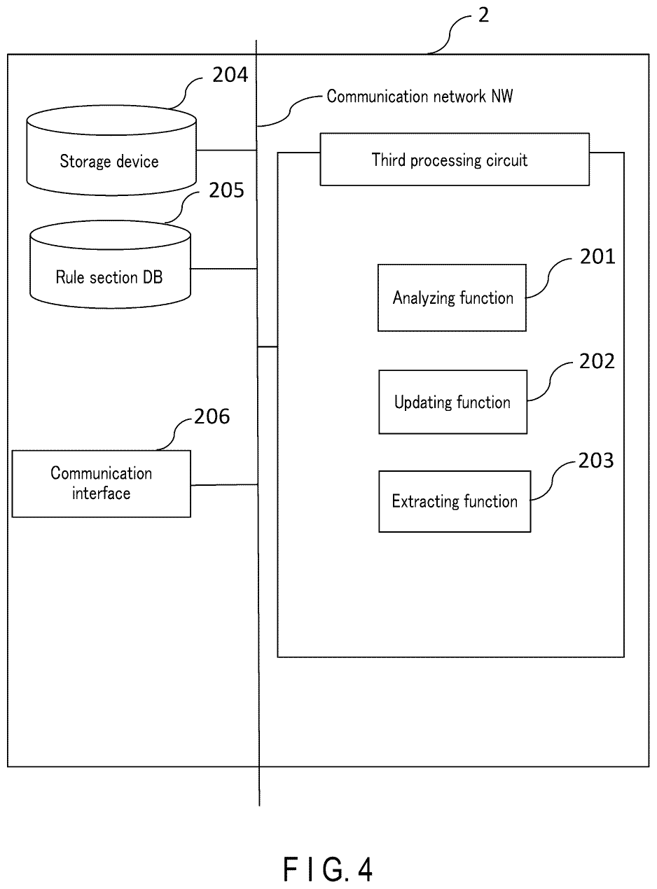

FIG. 4 is a block diagram showing an arrangement example of the feedback apparatus 2 according to the present embodiment. As shown in FIG. 4, the feedback apparatus 2 includes the third processing circuit, the storage device 204, a rule section DB 205, and a communication interface 206.

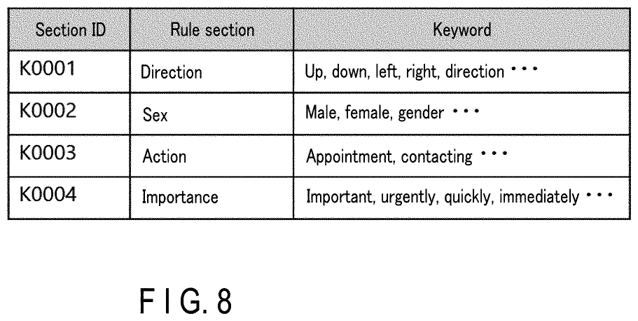

The rule section DB 205 is a relational database configured by a table like that shown in FIG. 8. The configuration of the database is not limited to a relational database. For example, the configuration may be a hierarchical database or network-type database.

The third processing circuit includes a CPU and a processor such as a GPU. In order to add, delete, or change warning rule information in the warning rule DB 507 of the radiogram interpretation report preparation support apparatus 5, the third processing circuit causes the processor to activate the feedback program installed in the storage device 204 or the like to implement an analyzing function 201, an updating function 202, an extracting function 203, and the like. Note that the functions 201 to 203 each need not always be implemented by a single processing circuit. Each processing circuit may be formed by combining a plurality of independent processors, and each processor may implement each of the functions 201 to 203 by executing a program. Alternatively, the analyzing function 201 and the updating function 202 each may be a program as a module constituting the radiogram interpretation preparation program or may be an individual program. These programs are stored in the storage device 204.

The medical information processing apparatus may be the feedback apparatus 2, the radiogram interpretation report preparation support apparatus 5, or the radiogram interpretation report preparation apparatus 4, or a combination of them.

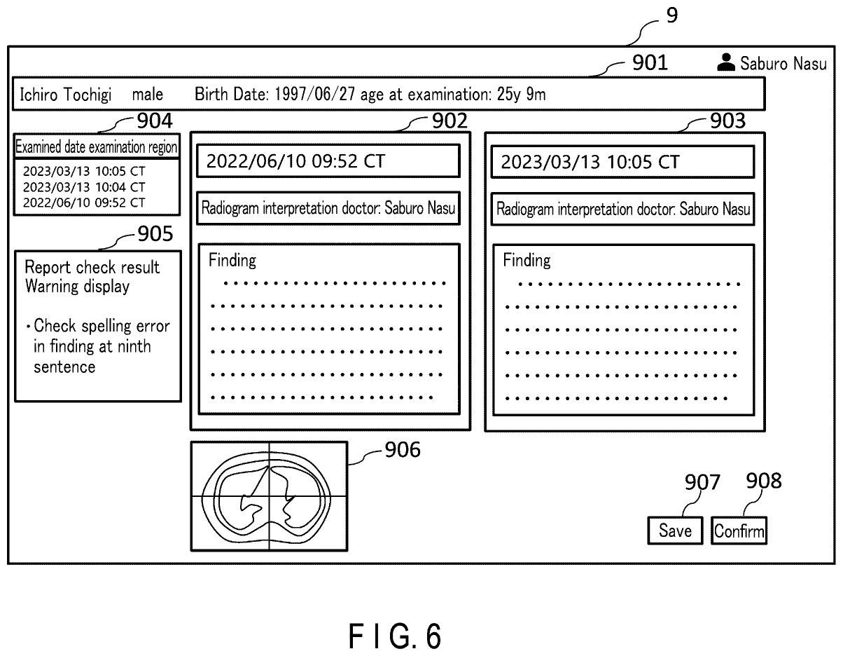

FIG. 6 shows an example of the display of a screen on the radiogram interpretation apparatus 3 performed by the radiogram interpretation report preparation apparatus 4 and the radiogram interpretation report preparation support apparatus 5. For example, the radiogram interpretation doctor writes a finding in a current radiogram interpretation report 903 upon checking a past radiogram interpretation report 902 and a key image 906. For example, upon pressing a save instruction button 907, the radiogram interpretation doctor analyzes the radiogram interpretation report using the radiogram interpretation report preparation support apparatus 5 and displays warning information 905 using the radiogram interpretation apparatus 3. The timing of the analysis of a radiogram interpretation report by the radiogram interpretation report preparation support apparatus 5 is not limited to after the pressing of the save instruction button 907. The analysis may be performed when the radiogram interpretation doctor writes a radiogram interpretation report or when the radiogram interpretation doctor pastes a key image on a radiogram interpretation report.

FIG. 7 shows a detailed example of the warning rule DB 507 of the radiogram interpretation report preparation support apparatus 5. The rule information of each warning information is managed by a rule ID. For example, rule ID “R1001” is rule information for displaying the warning message “check image and left and right of findings” when the radiogram interpretation doctor captures an image of the lungs in a CT examination and saves a corresponding report.

FIG. 8 shows a detailed example of the rule section DB 205 of the feedback apparatus 2. The extracting function 203 of the feedback apparatus 2 extracts a rule section based on a keyword of the warning message set in rule information. For example, if there is a keyword concerning a direction, such as “up”, “down”, “left”, or “right”, in the warning message of the rule information of section ID “K0001”, the extracting function 203 of the feedback apparatus 2 extracts the rule section of the rule information as a direction. Rule sections include, for example, information concerning sexes, actions, importances, and the like in addition to directions. A rule section is extracted when, for example, rule information is registered in the warning rule DB 507. Note, however, that the timing of the extraction of a rule section is not limited to when the operator registers rule information. When the control function 503 of the radiogram interpretation report preparation support apparatus 5 displays a warning message, the extracting function 203 of the feedback apparatus extracts a rule section.

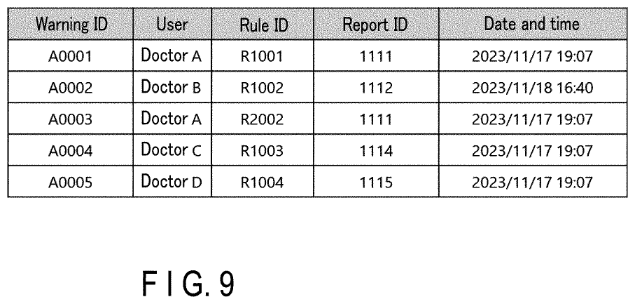

FIG. 9 is a detailed example of a display history information DB. For example, the radiogram interpretation report preparation support apparatus 5 displays history information indicating when warning information concerning an inappropriate description was displayed, to which report which rule information was applied, and for whom the warning message was displayed. For example, display history information with warning ID “A0001” represents history information indicating that the user was doctor A, the rule ID was R1001, the report ID was 1111, and the display date was Nov. 17, 2023. The history information is not limited to a database and may be a log that records and accumulates events in chronological order. The display history information is a specific example of warning history information.

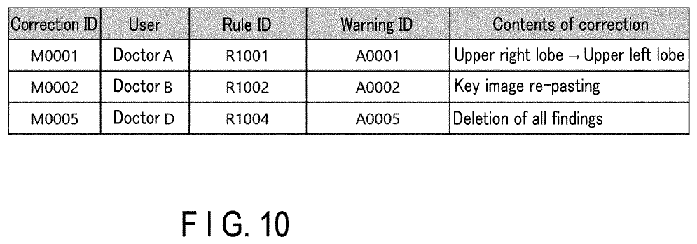

FIG. 10 shows one of specific examples of a radiogram interpretation report correction history information DB. The control function 503 of the radiogram interpretation report preparation support apparatus 5 displays a warning message for warning the radiogram interpretation doctor on a display screen of the display of the radiogram interpretation report preparation apparatus. This makes the radiogram interpretation doctor correct the report. For example, correction history information with correction ID “M0001” represents history information with warning ID “A0001” which has undergone correction of text information “upper right lobe” of the text information included in a finding column of a radiogram interpretation report into text information “upper left lobe”.

Details of correction concerning the display of warning information are, for example, writing error correction, finding addition, finding deletion, and key image re-pasting.

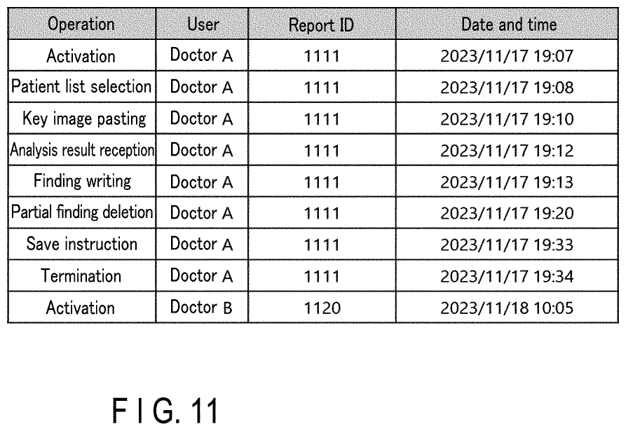

FIG. 11 shows one of specific examples of the operation history information DB 408. The operation history information DB 408 stores information recording which user, that is, which operator performed which operation at which date. For example, this information records that a given operator activated the radiogram interpretation report preparation apparatus 4, selected an examination list, pasted a key image on a radiogram interpretation report, imported an examination result, wrote a finding, deleted part of the finding, pressed the save instruction button 907, and pressed a confirm instruction button 908. The operation history information DB 408 is not limited to a database and may be a log that records and accumulates events in chronological order.

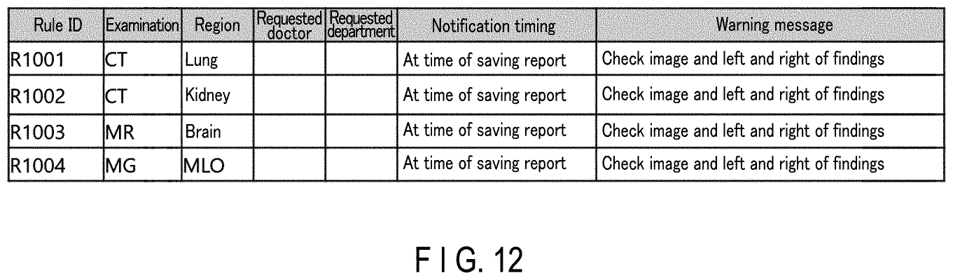

FIG. 12 shows one of specific examples of the DB 507 of rule information added by the updating function 202 of the feedback apparatus 2. Rule information with rule ID “R1001” is originally present in, for example, the warning rule DB 507. Rule information with rule ID “R1002”, rule information with rule ID “R1003”, and rule information with rule ID “R1004” each are, for example, added rule information. The rule information with rule ID “R1002”, the rule information with rule ID “R1003”, and the rule information with rule ID “R1004” are specific examples of rule information to be added, which belong to rule section “direction” and differ in examination, examination region, and notification timing.

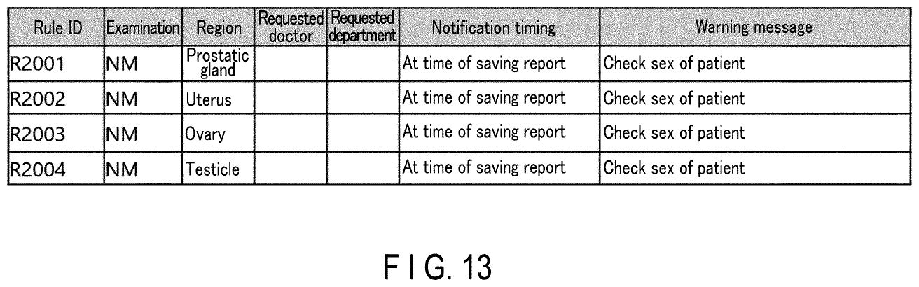

FIG. 13 shows one of specific examples of a rule information DB added by the updating function 202 of the feedback apparatus 2. Rule information with rule ID “R2001” is originally present in, for example, the warning rule DB 507. Rule information with rule ID “R2002”, rule information with rule ID “R2003”, and rule information with rule ID “R2004” each are, for example, added rule information. The rule information with rule ID “R2002”, the rule information with rule ID “R2003”, and the rule information with rule ID “R2004” are examples of rule information concerning sex.

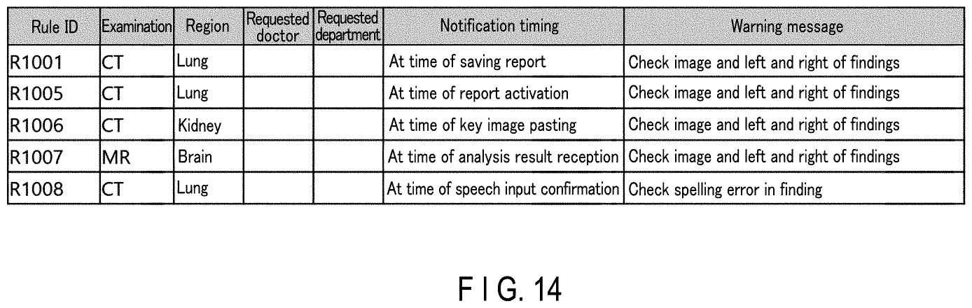

FIG. 14 shows one of specific examples of a rule information DB added by the updating function 202 of the feedback apparatus 2. Rule information with rule ID “R1001” is originally present in, for example, the warning rule DB 507. Rule information with rule ID “R1005”, rule information with rule ID “R1006”, rule information with rule ID “R1007”, and rule information with rule ID “R1008” each are, for example, added rule information. FIG. 14 shows an example of adding rule information concerning the notification timing at which warning information is displayed.

For example, the updating function 202 of the feedback apparatus 2 changes rule information often configured to display a warning message at the time of saving a radiogram interpretation report into rule information configured to display warning information for raising caution during the preparation of a radiogram interpretation report. The analyzing function 201 specifies rule information often configured to display a warning message at the time of saving a radiogram interpretation report. The updating function 202 of the feedback apparatus 2 changes, for example, rule information often configured to display warning information based on a difference between an examination image and the contents of a finding into rule information configured to display warning information for raising caution at the time of key image pasting or analysis result reception. In addition, for example, the updating function 202 of the feedback apparatus 2 changes rule information often configured to display warning information concerning erroneous conversion by speech input into rule information configured to display warning information for raising caution at the time of speech input confirmation. That is, the updating function 202 changes rule information so as to output warning information during the preparation of a radiogram interpretation report.

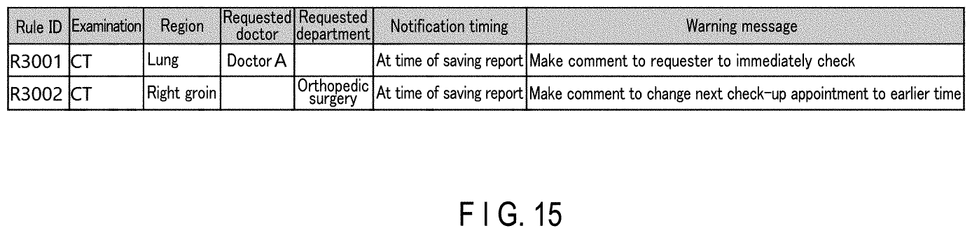

FIG. 15 shows one of specific examples of a DB of rule information added by the updating function 202 upon analysis of history information concerning a radiogram interpretation report from a medical treatment system 6 by the analyzing function 201 of the feedback apparatus. Rule information with rule ID “R3001” and rule information with rule ID “R3002” each are added rule information. For example, the rule information with rule ID “R3001” and the rule information with rule ID “R3002” each are an example of addition of rule information prompting to send a radiogram interpretation report upon attaching a caution or comment to the report when the radiogram interpretation report preparation apparatus 4 delivers the radiogram interpretation report to a medical treatment department. For example, to deliver a radiogram interpretation report is to make the radiogram interpretation report preparation apparatus 4 save the radiogram interpretation report and deliver it by Web delivery. Radiogram interpretation reports may be saved in the form of PDF (Portable Document Format) file or XML (Extensible Markup Language) file.

An operation example of the feedback apparatus 2 and an example of a processing procedure will be described below. Note that a radiogram interpretation report according to the present embodiment may concern any region, and any type of medical image obtained by any type of medical image diagnosis apparatus can be a medical image targeted for radiogram interpretation. Accordingly, the radiogram interpretation report preparation support apparatus 5 may analyze any types of radiogram interpretation reports.

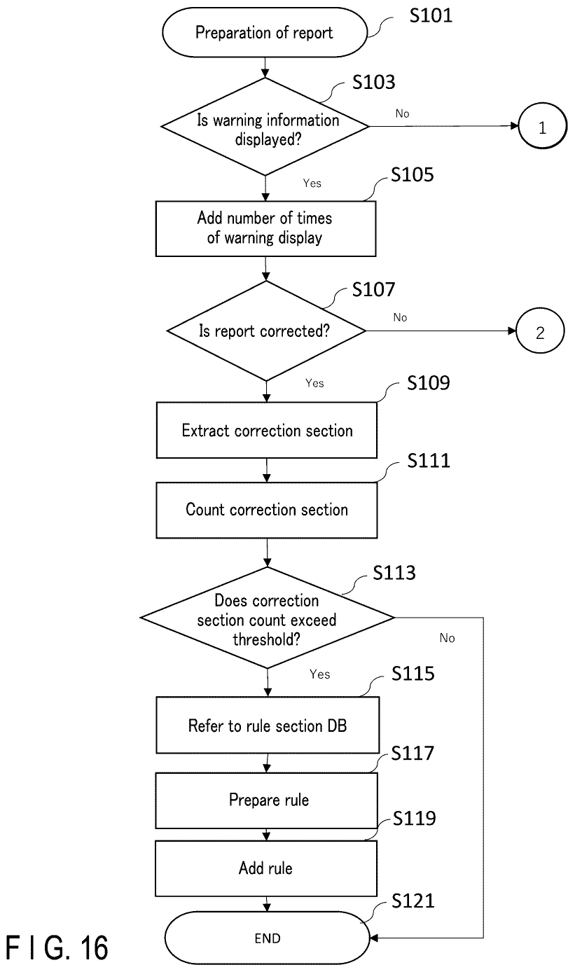

FIG. 16 is a flowchart in which the analyzing unit of the feedback apparatus 2 analyzes each history information, and the updating function 202 adds rule information to the warning rule DB 507 in the radiogram interpretation report preparation support apparatus 5. Each history information includes display history information, the correction history information of a radiogram interpretation report, the operation history information of the radiogram interpretation report preparation apparatus 4, radiogram interpretation report reference history information in the electronic medical chart system of the medical treatment system 6, radiogram interpretation report read management history information, check-up appointment history information, examination appointment history information, and prescription history information.

### Processing Procedure

### Step S101

The radiogram interpretation doctor prepares a radiogram interpretation report and presses the save button. The analyzing function 201 of the feedback apparatus 2 then starts analyzing history information. That is, the analyzing function 201 analyzes warning history information as history information indicating that warning information was output, warning history information indicating that no warning information was output, and correction history information as history information of correction of the radiogram interpretation report.

### Step S103

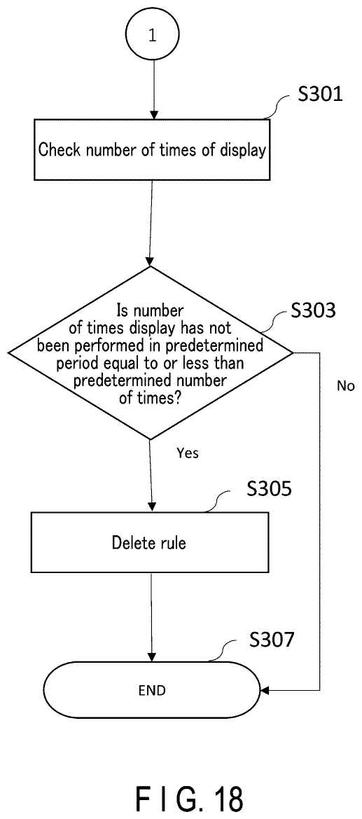

The morphological analysis function 501 and the pattern matching processing function 502 of the radiogram interpretation report preparation support apparatus 5 analyze whether there is any inappropriate description in the prepared radiogram interpretation report. If there is an inappropriate description, the control function 403 of the radiogram interpretation report preparation apparatus 4 displays warning information on the display 410. If warning information is displayed, the process advances to step S105. If no warning information is displayed, the process advances to step S301 in FIG. 18.

### Step S105

The analyzing function 201 of the feedback apparatus 2 increments a warning display counter by +1 with respect to each rule information that made the radiogram interpretation report preparation support apparatus 5 display warning information, thereby counting the number of times a warning message was displayed in accordance with each rule information. The warning display counter is stored as the value of the storage device 204 of the feedback apparatus 2. The analyzing function 201 of the feedback apparatus 2 refers to the stored value.

### Step S107

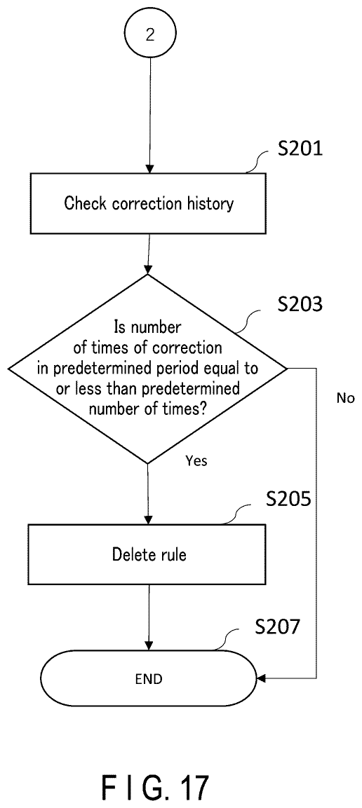

The analyzing function 201 of the feedback apparatus 2 refers to the correction history information DB 407 to analyze whether there was any correction of each rule information that made the radiogram interpretation report preparation support apparatus 5 display warning information. If there was any correction history information corresponding to each rule information that made warning information be displayed, the process advances to step S109. If there was no correction history information, the process advances to step S201 in FIG. 17. For example, the analyzing function 201 of the feedback apparatus 2 analyzes that there is correction history information with M0001 in the correction history information DB 407 in FIG. 10 with respect to rule ID “R1001” in the display history information DB 504 in FIG. 9.

### Step S109

If there is any correction history information, the analyzing function 201 of the feedback apparatus 2 analyzes the contents of the correction in the correction history information stored in the correction history information DB 407. The extracting function 203 extracts the analysis result as a correction section and collates it with a rule section. For example, the analyzing function 201 of the feedback apparatus 2 analyzes that text information of “upper right lobe” of the text information included in the finding column of the radiogram interpretation report corresponding to M0001 of the correction history information DB 407 in FIG. 10 was corrected to text information of “upper left lobe” and also analyzes that this corresponds to a correction section concerning direction. Referring to FIG. 8, the analyzing function 201 of the feedback apparatus 2 analyzes that the rule section of the rule information corresponding to R0001 is direction and also analyzes that the radiogram interpretation doctor saw and corrected a displayed warning message.

### Step S111

The analyzing function 201 of the feedback apparatus 2 increments the rule section correction counter set for each rule section with respect to the correction section extracted in step S109. For example, since the correction section of the correction history information corresponding to M0001 is direction, the analyzing function 201 of the feedback apparatus 2 increments the rule section correction counter for direction by +1. The rule section correction counter is stored as the value of the storage device 204 of the feedback apparatus 2. The analyzing function 201 of the feedback apparatus 2 refers to the stored value.

### Step S113

The analyzing function 201 of the feedback apparatus 2 determines whether the value of the rule section correction counter counted in step S111 exceeds a set threshold. If the value exceeds the threshold, the process advances to step S115. The warning display counter, the rule section correction counter, and rule information exceeding each threshold constitute target rule information. That is, the analyzing function 201 specifies, as target rule information, rule information for which the number of times a warning message has been output during a predetermined period is equal to or more than a predetermined number of times and based on which a radiogram interpretation report has been corrected in accordance with a warning message is equal to or more than a predetermined number of times. If the rule information does not exceed the threshold, the process advances to step S121 to terminate this processing. A threshold may be stored in the storage device 204 so as to allow the operator to arbitrary set a threshold or may be set in advance in the apparatus.

### Step S115

The analyzing function 201 of the feedback apparatus 2 refers to the rule section DB 205 to acquire a rule section corresponding to a rule section correction counter that has exceeded the threshold.

### Step S117

The updating function 202 of the feedback apparatus 2 generates a rule section and similar rule information which were referred to in step S115. For example, in the case of the rule section corresponding to direction, similar rule information concerning direction is generated. Pieces of similar rule information of direction are pieces of rule information equal in warning message but different in examination technique, examination region, or notification timing, like pieces of rule information R1002, R1103, and R1104 with respect to rule information R1001 in FIG. 12. In this case, R1001 corresponds to target rule information. Referring to FIG. 12, regions of similar rule information are organs each having left and right parts in the human body. That is, referring to FIG. 12, such regions include kidney, brain, and breast. In addition, an examination technique is selected from past display history information. Assume that a kidney CT image was interpreted in the past, and warning information was displayed with respect to the corresponding radiogram interpretation report. In this case, the updating function 202 of the feedback apparatus 2 selects CT as examination information corresponding to kidney and adds the information as similar rule information. Alternatively, the analyzing function 201 of the feedback apparatus 2 may acquire information of a medical device installed in the hospital from the RIS, and the updating function 202 may select the information as examination information and add it as similar rule information. In addition, all combinations of rule information of examination information and regions may be added as similar rule information.

### Step S119

The updating function 202 of the feedback apparatus 2 adds the rule information generated in step S117 to the warning rule DB 507 of the radiogram interpretation report preparation support apparatus 5.

### Step S121

This processing is terminated.

The above is an example in which the correction history information and display history information of a radiogram interpretation report are analyzed in the operation procedure from step S101 to step S121, and new similar rule information is added to the warning rule DB 507. However, new similar rule information may be added to the warning rule DB 507 by analyzing the operation history information of the radiogram interpretation report apparatus, the history information of an apparatus on the medical treatment system 6 side, and the like in addition to the correction history information of the radiogram interpretation report. The analyzing function 201 analyzes history information concerning a radiogram interpretation report in the medical treatment system 6. The updating function 202 updates rule information for outputting warning information concerning the correction of the radiogram interpretation report based on the analysis result obtained by the analyzing function 201.

If warning information is frequently displayed and a radiogram interpretation report is frequently corrected at a specific region depending on sex, the updating function 202 of the feedback apparatus adds rule information for displaying warning information at another region which is a specific region depending on sex. For example, as shown in FIG. 13, rule information with rule ID “R2001” is originally present in the warning rule DB 507. Rule information with rule ID “R2002”, rule information with rule ID “R2003”, and rule information with rule ID “R2004” each are rule information added concerning a specific region depending on sex.

The feedback apparatus 2 adds rule information to the warning rule DB 507. Since an inappropriate description concerning fail to display warning information can be detected before the addition, it is possible to improve the accuracy of detection of inappropriate descriptions in the radiogram interpretation report preparation support apparatus 5.

By adding rule information to the warning rule DB 507, the feedback apparatus 2 can reduce the trouble of maintenance of adding rule information, which has been manually performed.

This will customize the warning rule DB 507 for each hospital or each radiogram interpretation doctor, thereby improving the accuracy of detection of inappropriate descriptions in the radiogram interpretation report preparation support apparatus 5.

FIG. 17 is a flowchart in which the analyzing unit of the feedback apparatus 2 analyzes each history information, and the updating function 202 deletes information concerning rule information stored in the warning rule DB 507 of the radiogram interpretation report preparation support apparatus 5. FIG. 17 shows a case where the radiogram interpretation report preparation support apparatus 5 displays warning information.

### Step S201

The analyzing function 201 of the feedback apparatus 2 refers to the correction history information DB 407 with respect to each rule information that made warning information be displayed.

### Step S203

The analyzing function 201 of the feedback apparatus 2 refers to the correction history information DB 407. If the analyzing function 201 specifies, as the first deletion rule information, each rule information that made warning information be displayed for which the number of times a radiogram interpretation report has been corrected during a predetermined period is equal to or less than a predetermined number of times, the process advances to step S205. If the analyzing function 201 analyzes that the number of times a radiogram interpretation report has been corrected is larger than a predetermined number of times with respect to each rule information that made warning information be displayed in a predetermined period, the process advances to step S207. The predetermined period may be one week or one month. The predetermined number of times is the number of times of analysis of a description in a radiogram interpretation report by the radiogram interpretation report preparation support apparatus 5. The operator may arbitrarily set a predetermined period and a predetermined number of times. Alternatively, a predetermined period and a predetermined number of times may be set in advance in the apparatus. A predetermined period and a predetermined number of times are stored as values of the storage device 204, and the analyzing function 201 of the feedback apparatus 2 refers to the stored values.

### Step S205

The updating function 202 of the feedback apparatus 2 deletes the specified first deletion rule information from the warning rule DB 507.

### Step S207

This processing is terminated.

The present embodiment can reduce the display of unnecessary warning information by deleting unnecessary rule information from the warning rule DB 507 of the radiogram interpretation report preparation support apparatus 5. This makes it possible to reduce the capacity of the warning rule DB 507 and improve the speed of detecting inappropriate descriptions by the radiogram interpretation report preparation support apparatus 5. Unnecessary warning information is warning information that has not been corrected by the radiogram interpretation doctor in spite of the fact that warning information is displayed.

The feedback apparatus 2 can reduce the trouble of maintenance of deleting rule information, which has been a manual operation, by deleting rule information in the warning rule DB 507.

The example of the operation procedure shown in FIG. 17 is an example in which although the radiogram interpretation report preparation support apparatus 5 has displayed warning information, the radiogram interpretation doctor has not corrected the radiogram interpretation report based on the display of the warning information, and hence the analyzing function 201 of the feedback apparatus 2 determines that the rule information is unnecessary, and the updating function 202 deletes the rule information. Determining that the rule information is unnecessary and deleting the rule information will inhibit the unnecessary warning message from being displayed and improve the efficiency of a correcting operation by the radiogram interpretation doctor.

FIG. 18 is a flowchart in which the analyzing function 201 of the feedback apparatus 2 analyzes each history information, and the updating function 202 deletes rule information from the warning rule DB 507 of the radiogram interpretation report preparation support apparatus 5 and shows a case wherein the radiogram interpretation report preparation support apparatus 5 displays no warning information.

### Step S301

The analyzing function 201 of the feedback apparatus 2 refers to the warning display counter stored in the storage device 204 with respect to each rule information that did not make warning information be displayed by the radiogram interpretation report preparation support apparatus 5.

### Step S303

If the analyzing function 201 of the feedback apparatus 2 specifies, as the second deletion rule, rule information for which the number of times warning information has been displayed is equal to or less than a predetermined number of times in a predetermined time, the process advances to step S305. If the analyzing function 201 analyzes that the number of times warning information has been displayed is larger than the predetermined number of times in the predetermined period, the process advances to step S307. The predetermined period may be one week or one month. The predetermined number of times is the number of times of analysis of a description in a radiogram interpretation report by the radiogram interpretation report preparation support apparatus 5. The operator may arbitrarily set a predetermined period and a predetermined number of times. Alternatively, a predetermined period and a predetermined number of times may be set in advance in the apparatus. A predetermined period and a predetermined number of times are stored as values of the storage device 204, and the analyzing function 201 of the feedback apparatus 2 refers to the stored values. History information that did no make warning information be displayed is information that did not make warning information be displayed by the radiogram interpretation report preparation support apparatus 5 with respect to each rule information.

### Step S305

The updating function 202 of the feedback apparatus 2 deletes the specified second deletion rule information from the warning rule DB 507.

### Step S307

This processing is terminated.

Deleting rule information of unnecessary warning information makes it possible to reduce the storage capacity constituting the warning rule DB 507 and shorten the processing times of the morphological analysis function 501 of the radiogram interpretation report preparation support apparatus 5 and the pattern matching processing function 502.

An operation of making the pattern matching processing function 502 of the radiogram interpretation report preparation support apparatus 5 not use rule information may be regarded as an operation of deleting rule information. For example, the apparatus may hold information for managing whether to use each rule information in the warning rule DB 507. Based on rule information that is not used, the pattern matching processing function 502 does not use the rule information. If the analyzing function 201 of the feedback apparatus 2 analyzes that given rule information is unnecessary, the updating function 202 may update information for managing whether to use the rule information into information that makes the information unusable. Alternatively, the operator may set the rule information as usable or unusable information.

### Second Embodiment

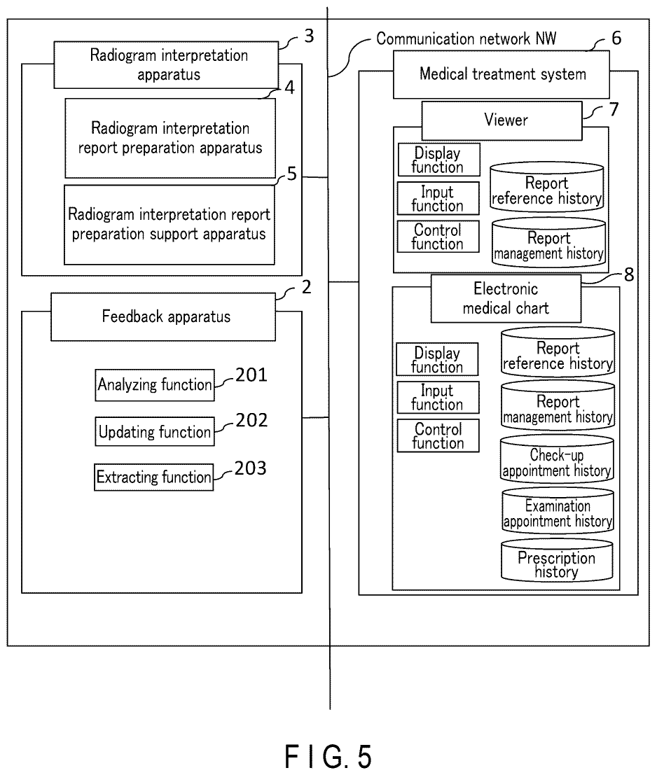

According to a second embodiment, a feedback apparatus 2 may be linked with a medical treatment system 6. The medical treatment system 6 includes a viewer 7 on which various types of information of a patient like those shown in FIG. 5 are browsed and an electronic medical chart 8 that stores the results of recording check-up on the patient, examination results, and medication instruction history information. For example, an analyzing function 201 of the feedback apparatus 2 in FIG. 5 analyzes history information on the viewer 7 and the electronic medical chart 8 which are used by a doctor in a medical treatment department to check information about the patient. History information on the viewer 7 includes the reference history information of a radiogram interpretation report by a doctor in the medical treatment department and management history information (radiogram interpretation report read management information) of the radiogram interpretation report by the doctor in the medical treatment department. History information on the electronic medical chart 8 includes the reference history information of a radiogram interpretation report by a doctor in the medical treatment department, management history information (radiogram interpretation report read management information) of the radiogram interpretation report by the doctor in the medical treatment department, check-up appointment history information, examination appointment history information, and prescription history information.

Information about the elapsed time from when a radiogram interpretation report is prepared or saved to when the report is browsed exists as history information of the viewer 7. The analyzing function 201 of the feedback apparatus 2 analyzes the history information of the viewer 7 and specifies a doctor who took a long time until he/she browsed the radiogram interpretation report. For example, if the time from when a radiogram interpretation report is prepared to when the report is browsed exceeds a predetermined time, the time until the radiogram interpretation report is browsed is regarded as long. The analyzing function 201 analyzes history information indicating that the time from when a radiogram interpretation report is prepared to when the report is browsed exceeds the predetermined time. Note that a predetermined time may be arbitrarily set by the operator or set in advance in the apparatus. As indicated by rule ID “R3001” in FIG. 15, if the doctor in the medical treatment department who made a request is doctor A, an updating function 202 of the feedback apparatus 2 adds rule information configured to output the warning information “make comment to requester to immediately check”. The updating function 202 adds a rule for prompting to make comment to the requester to immediately check the radiogram interpretation report.

Letting the feedback apparatus 2 add rule information for displaying the warning message “make comment to requester to immediately check” makes it possible to shorten the time from when the radiogram interpretation report is prepared in a radiology department to when a doctor in the medical treatment department browses the report.

If, for example, a given patient consults an orthopedic surgeon about the suspicion of right hip joint fracture, the orthopedic surgeon requests the radiology department to image the right inguinal region. When the radiology department images the right inguinal region, the captured medical image sometimes includes the kidney. The radiogram interpretation doctor finds the suspicion of tumor, writes the corresponding finding in a radiogram interpretation report, and delivers the radiogram interpretation report to the orthopedic surgeon as the requester by saving the report. Subsequently, the patient is expected to consult the urology department. However, the orthopedic surgeon doctor may finish the consultation upon diagnosing only the right hip joint fracture without transferring the patient to the urology department. In addition, the orthopedic surgeon doctor may overlook a finding “there is a suspicion of tumor in the kidney” in the radiogram interpretation report.

The orthopedic surgeon doctor can transfer the patient to the urology department by making the feedback apparatus 2 add rule information for displaying the warning information “make comment to change next check-up appointment to earlier time” corresponding to rule ID “R3002” in FIG. 15. The updating function 202 adds a rule prompting a comment to schedule an appointment with an appropriate medical treatment department if the medical condition input to the radiogram interpretation report does not match the requested medical treatment department.

There is no limitation on the timing when the analyzing function 201 of the feedback apparatus 2 starts analyzing history information and the updating function 202 performs addition, deletion, changing, and the like of rule information. Such operations may be performed as needed or at periodic timings designated by the operator or timings designated by the operator.

Note that the operator is assumed to be a radiogram interpretation doctor, but this does not exclude healthcare personnel such as another doctor, a nurse, or technician or another third person.

In a case where the technical idea of the present embodiment is implemented by a medical information processing system, the medical information processing system including a radiogram interpretation report preparation apparatus and a radiogram interpretation report preparation support apparatus communicably connected to each other via a network includes an analyzing unit that analyzes history information concerning a radiogram interpretation report and an updating unit that updates rule information for outputting warning information concerning the correction of a radiogram interpretation report based on the analysis result obtained by the analyzing unit. Processing and effects in the medical information processing system are the same as those in the embodiment, and hence a description thereof will be omitted.

In a case where the technical idea of the present embodiment is implemented by a medical information processing method, the medical information processing method performs analysis processing of analyzing history information concerning a radiogram interpretation report and updates rule information for outputting warning information concerning the correction of a radiogram interpretation report based on the analysis result obtained by the analysis processing. Processing procedures and effects in the medical information processing method are the same as those in the embodiment, and hence a description thereof will be omitted.

In a case where the technical idea of the present embodiment is implemented by a medical information processing program, the medical information processing program causes a computer to implement analyzing history information concerning a radiogram interpretation report and updating rule information for outputting warning information concerning the correction of the radiogram interpretation report based on the analysis result. Processing procedures and effects in the medical information processing program are the same as those in the embodiment, and hence a description thereof will be omitted.

Note that various data handled in this specification are typically digital data.

The term “processor” used in the above description means a circuit such as a CPU, GPU, ASIC (Application Specific Integrated Circuit), SPLD (Simple Programmable Logic Device), CPLD (Complex Programmable Logic Device), or FPGA (Field Programmable Gate Array). The processor reads out a program stored in a storage circuit and executes the program to implement the corresponding function. Note that a program may be directly incorporated in a circuit of the processor instead of being saved in a storage circuit. In this case, the processor reads out the program incorporated in the circuit and executes the program to implement the corresponding function. In contrast to this, the function corresponding to the program may be implemented by a combination of logic circuits instead of executing the program. Note that each processor according to the present embodiment may be formed as one processor by combining a plurality of independent circuits to implement the corresponding function as well as being formed as a single circuit. In addition, a plurality of constituent elements in FIG. 1 may be integrated into one processor to implement the corresponding function.

### Third Embodiment

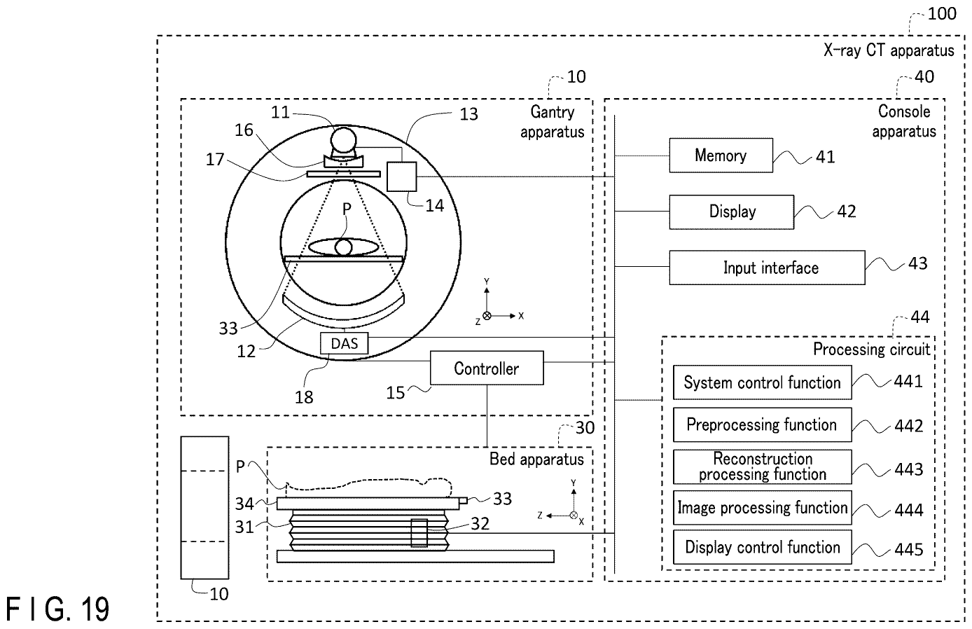

FIG. 19 shows an arrangement example of an X-ray CT apparatus 100 according to a third embodiment. The X-ray CT apparatus 100 includes a gantry apparatus 10, a bed apparatus 30, and a console apparatus 40. For the sake of descriptive convenience, the plurality of gantry apparatuses 10 are shown. A three-dimensional orthogonal coordinate system is defined with respect to the gantry apparatus 10 and the bed apparatus 30. The three-dimensional orthogonal coordinate system has an X-axis, a Y-axis, and a Z-axis which are orthogonal to each other. The Z-axis direction is defined as the direction of the rotation axis of a rotating frame 13 (or the longitudinal direction of a top plate 33 of the bed apparatus 30) in a non-tilt state. The X-axis direction is defined as an axial direction orthogonal to the Z-axis direction and horizontal to the floor surface. The Y-axis direction is defined as an axial direction orthogonal to the Z-axis direction and perpendicular to the floor surface.

### Gantry Apparatus

The gantry apparatus 10 is an apparatus for imaging a subject P. The gantry apparatus 10 includes an X-ray tube 11, an X-ray detector 12, a rotating frame 13, a high voltage generator 14, a controller 15, a wedge 16, a collimator 17, and a DAS 18.

The X-ray tube 11 is a vacuum tube that generates X-rays. The X-ray tube 11 generates X-rays by emitting thermal electrons from a cathode filament to an anode target using the high voltage applied from the high voltage generator 14. The X-ray tube 11 may be a rotating anode X-ray tube that generates X-rays by irradiating the rotating anode with thermal electrons.

The wedge 16 is a filter that adjusts the dose of X-rays. The wedge 16 transmits and attenuates X-rays applied from the X-ray tube 11 such that the dose of X-rays applied from the X-ray tube 11 to the subject P matches a predetermined distribution. The wedge 16 may be an aluminum filter processed to match a predetermined target angle (or a predetermined thickness).

The collimator 17 is a lead plate that limits the irradiation range of X-rays. The collimator 17 forms a slit by using a combination of a plurality of lead plates. The collimator 17 limits the irradiation range of X-rays transmitted through the wedge 16 through the slit. The collimator 17 is also called an “X-ray aperture”.

The X-ray detector 12 is a device that detects X-rays. The X-ray detector 12 detects the X-rays applied from the X-ray tube 11 and transmitted through the subject P. The X-ray detector 12 outputs an electrical signal corresponding the detected dose of X-rays to the DAS 18.

The high voltage generator 14 is a device that generates a high voltage. The high voltage generator 14 may be based on a transformer scheme (or inverter scheme). The high voltage generator 14 applies a generated high voltage to the X-ray tube 11.

The DAS 18 is a system (that is, a data acquisition system) that acquires data. The DAS 18 amplifies the electrical signal output from the X-ray detector 12. The DAS 18 generates projection data by converting the amplified electrical signal into a digital signal. The DAS 18 transmits the projection data to the console apparatus 40.

The rotating frame 13 is a rotatable annular frame. The rotating frame 13 supports the X-ray tube 11, the X-ray detector 12, the high voltage generator 14, the wedge 16, the collimator 17, and the DAS 18. The rotating frame 13 supports the X-ray tube 11 and the X-ray detector 12 so as to make them face each other. The rotating frame 13 rotates about the rotation axis (described above) under the control of the controller 15.

The controller 15 is an apparatus that executes various types of control. The controller 15 controls the gantry apparatus 10 and the bed apparatus 30 in accordance with input signals from the console apparatus 40. The controller 15 executes control to tilt the gantry apparatus 10, control to rotate the rotating frame 13, control to raise and lower the bed apparatus 30, and control to slide the top plate 33.

### Bed Apparatus

The bed apparatus 30 is an apparatus that moves the subject P while it is placed thereon. The bed apparatus 30 is installed to face the opening (also called the “bore”) of the gantry apparatus 10. The bed apparatus 30 includes a base 31, a driving apparatus 32, a top plate 33, and a support frame 34.

The base 31 is a housing that supports the support frame 34. The base 31 accommodates the driving apparatus 32. The base 31 is installed on the floor surface.

The driving apparatus 32 is an apparatus that drives the base 31 and the top plate 33. The driving apparatus 32 drives the base 31 and the top plate 33 in accordance with input signals from the controller 15 (or the console apparatus 40). The driving apparatus 32 drives the base 31 in the vertical direction (that is, the Y-axis direction). The driving apparatus 32 drives the top plate 33 in the longitudinal direction (that is, the Z-axis direction). The driving apparatus 32 may be a motor (or actuator).

The top plate 33 is a flat plate on which the subject P is placed. The top plate 33 is installed on the support frame 34.

The support frame 34 is a frame that supports the top plate 33. The top plate 33 is installed on the base 31.

### Console Apparatus

The console apparatus 40 is an apparatus (that is, a computer) that controls the gantry apparatus 10 and the bed apparatus 30. The console apparatus 40 includes a memory 41, a display 42, an input interface 43, and a processing circuit 44.

The memory 41 is a device that stores various data. The memory 41 stores projection data, reconstructed image data, and the like. The memory 41 may be a hard disk, optical disk, semiconductor memory, or the like. The memory 41 is an example of a storage unit.

Note that the memory 41 may store various data (or databases) provided in the medical information processing system 1 (see FIG. 1). More specifically, the memory 41 may store a report DB 404, a storage device 405, examination information 406, a correction history information DB 407, an operation history information DB 408, and the like as various data (or databases) provided in the radiogram interpretation report preparation apparatus 4 (see FIG. 2). The memory 41 may store a display history information DB 504, a storage device 505, a medical knowledge dictionary DB 506, a warning rule DB 507, and the like as various data (or databases) provided in the radiogram interpretation report preparation support apparatus 5 (see FIG. 3). The memory 41 may store a storage device 204, a rule section DB 205, and the like as various data (or databases) provided in the feedback apparatus 2 (see FIG. 4).

Note that the memory 41 may store various data (or databases) provided in the medical treatment system 6 (see FIG. 5). More specifically, the memory 41 may store a report reference history, a report management history, and the like as various data (or databases) provided in a viewer 7. The memory 41 may store a report reference history, a report management history, a check-up appointment history, an examination appointment history, prescription history, and the like as various data (or databases) provided in an electronic medical chart 8.

The display 42 is a device that displays various types of information. The display 42 displays medical images, GUIs (Graphical User Interfaces), and the like. The display 42 may be a liquid crystal display, CRT (Cathode Ray Tube) display, tablet terminal, or the like. The display 42 is an example of a display unit.

The input interface 43 is an interface that receives various types of inputs. The input interface 43 receives acquisition conditions for projection data, reconstruction conditions for CT images, image processing conditions for postprocessing images, and the like. The input interface 43 converts a received input into an electrical signal and outputs the electrical signal to the processing circuit 44. The input interface 43 may be a mouse, keyboard, trackball, switches, buttons, joystick, table terminal, or the like. The input interface 43 is an example of an input unit.

The processing circuit 44 is a circuit that executes various types of processing. The processing circuit 44 controls the overall operation of the X-ray CT apparatus 100. The processing circuit 44 implements a system control function 441, a preprocessing function 442, a reconstruction processing function 443, an image processing function 444, and a display control function 445. The processing circuit 44 is an example of a processing unit.

The system control function 441 is a function of controlling various types of functions of the processing circuit 44. The system control function 441 controls the processing circuit 44 based on inputs received from the operator via the input interface 43. The system control function 441 is an example of a system control unit.

The preprocessing function 442 is a function of executing various types of preprocessing. The preprocessing function 442 generates preprocessed projection data by executing preprocessing (for example, logarithmic transformation, offset correction, sensitivity correction, or beam hardening correction) with respect to the projection data transmitted from the DAS 18. The preprocessing function 442 is an example of a preprocessing unit.

The reconstruction processing function 443 is a function of executing various types of reconstruction processing. The reconstruction processing function 443 generates a CT image by executing reconstruction processing (for example, filtered back projection or successive approximation reconstruction) with respect to preprocessed projection data. The reconstruction processing function 443 is an example of a reconstruction processing unit.

The image processing function 444 is a function of executing various types of image processing. The image processing function 444 generates display image data by executing image processing (for example, MPR (Multi Planar Reconstruction), MIP (Maximum Intensity Projection), or VR (Volume Rendering)) with respect to CT image data. The image processing function 444 is an example of an image processing unit.

The display control function 445 is a function of controlling various types of display modes. The display control function 445 generates display image data having undergone window processing by executing window processing with respect to display image data. The display control function 445 causes the display 42 to display the display image data having undergone the window processing. The display control function 445 is an example of a display control unit.

Note that the processing circuit 44 may implement various types of functions provided in the medical information processing system 1 (see FIG. 1). More specifically, the processing circuit 44 may implement various types of functions (an analyzing function 201, an updating function 202, and an extracting function 203) provided in the feedback apparatus 2. The processing circuit 44 may implement various types of functions (an input function 401, a save instruction function 402, and a control function 403) provided in the radiogram interpretation report preparation apparatus 4. The processing circuit 44 may implement various types of functions (the morphological analysis function 501, the pattern matching processing function 502, and the control function 503) provided in the radiogram interpretation report preparation support apparatus 5.

For example, the processing circuit 44 may analyze history information concerning a radiogram interpretation report via the analyzing function 201. The processing circuit 44 may update rule information for outputting warning information concerning a radiogram interpretation report based on an analysis result of history information via the updating function 202.

Note that the processing circuit 44 may implement various types of functions provided in a medical treatment system 6 (see FIG. 5). More specifically, the processing circuit 44 may implement various types of functions (a display function, an input function, and a control function) provided in the viewer 7. The processing circuit 44 may implement various types of functions (a display function, an input function, and a control function) provided in the electronic medical chart 8.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising a processing circuit configured to:
analyze history information concerning a radiogram interpretation report; and
update rule information for outputting warning information concerning correction of the radiogram interpretation report based on an analysis result of the history information.

2. The apparatus according to claim 1, wherein the processing circuit is further configured to
analyze warning history information as history information for which the warning information has been output and correction history information as history information of correction of the radiogram interpretation report.

3. The apparatus according to claim 1, wherein the processing circuit is further configured to
analyze warning history information as history information for which the warning information has not been output and correction history information as history information of correction of the radiogram interpretation report.

4. The apparatus according to claim 2, wherein the processing circuit is further configured to:
specify, as target rule information, rule information for which a number of times the warning information has been output is not less than a predetermined number of times and a number of times a radiogram interpretation report has been corrected is not less than a predetermined number of times in a predetermined period; and
add, to a warning rule database, similar rule information that is identical to warning information of the target rule information and differs in at least one of the target rule information, region information, and notification timing of examination information.

5. The apparatus according to claim 2, wherein the processing circuit is further configured to:
specify, as first deletion rule information, rule information for which a number of times a radiogram interpretation report has been corrected in accordance with the warning information in a predetermined period is not more than a predetermined number of times; and
delete the first deletion rule information from a warning rule database.

6. The apparatus according to claim 3, wherein the processing circuit is further configured to:
specify, as second deletion rule information, rule information for which a number of times the warning information has been output in a predetermined period is not more than a predetermined number of times; and
delete the second deletion rule information from a warning rule database.

7. The apparatus according to claim 2, wherein the processing circuit is further configured to:
specify, based on the warning history information, warning information output a number of times not less than a predetermined number of times when the radiogram interpretation report is saved; and
update the rule information so as to output the warning information during preparation of the radiogram interpretation report.

8. The apparatus according to claim 1, wherein the processing circuit is further configured to
extract a rule section of the warning information when the rule information is registered in a warning rule database.

9. The apparatus according to claim 8, wherein the processing circuit is further configured to:
analyze correction history information;
extract an analysis result of the correction history information as a correction section; and
collate the correction section with the rule section.

10. The apparatus according to claim 1, wherein the processing circuit is further configured to:
analyze history information concerning a radiogram interpretation report in a medical treatment system; and
update rule information for outputting warning information concerning correction of a radiogram interpretation report based on an analysis result of the history information.

11. The apparatus according to claim 10, wherein history information concerning a radiogram interpretation report in the medical treatment system includes at least one of a viewer used in a medical treatment department, radiogram interpretation report reference in an electronic medical chart system, radiogram interpretation report read management, a check-up appointment, an examination appointment, and prescription.

12. The apparatus according to claim 11, wherein the processing circuit is further configured to:
analyze history information indicating that a time from when the radiogram interpretation report is prepared to when the radiogram interpretation report is browsed exceeds a predetermined time; and
add a rule for prompting to make a comment to a requester to immediately check the radiogram interpretation report based on an analysis result of the history information.

13. The apparatus according to claim 12, wherein the processing circuit is further configured to
add a rule for prompting to make a comment to schedule an appointment with an appropriate medical treatment department when a medical condition input to a radiogram interpretation report does not match a requested medical treatment department.

14. A medical information processing system including a radiogram interpretation report preparation apparatus and a radiogram interpretation report preparation support apparatus which are connected to each other so as to be communicable via a network, the system comprising
a processing circuit configured to:
analyze history information concerning a radiogram interpretation report; and
update rule information for outputting warning information concerning correction of the radiogram interpretation report based on an analysis result of the history information.

15. A medical information processing method comprising:
analyzing history information concerning a radiogram interpretation report; and
updating rule information for outputting warning information concerning correction of the radiogram interpretation report based on an analysis result of the history information.
